(19) United States
(12) Patent Application Publication
(10) Pub. No.: US 2008/0076121 A1
(43) Pub. Date: Mar. 27, 2008

(54) Microarray nuclease protection assay

(76) Inventor: Paul Kenneth Wolber, Los Altos Hills, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/525,986

(22) Filed: Sep. 22, 2006

(51) Int. Cl. C12Q 1/68; C12M 3/00

(52) U.S. Cl. 435/6; 435/287.2

(57) ABSTRACT

A method for microarray analysis of a sample for a target nucleic acid sequence including selecting a microarray comprising a set of oligonucleotide probes, contacting the set of oligonucleotide probes with a sample suspected of containing a target nucleic acid sequence under hybridization conditions to form a set of probe-target hybrids, contacting the set of probe-target hybrids with one or more single-stranded nucleases, removing the target nucleic acids from the remaining set of probe-target hybrids to expose the oligonucleotide probes remaining on the microarray, and labeling the remaining oligonucleotide probes, and analyzing the microarray for oligonucleotide probes hybridized to the target nucleic acid sequences.

### BACKGROUND

Gene-chip and other use of nucleic acid array technologies in molecular biology, biochemistry and biophysics rely on the hybridization of a nucleic acid molecule to another support-bound nucleic acid molecule. One goal of these techniques is to determine the presence and/or amounts of nucleic acid molecules containing nucleotide sequences of interest. In general, nucleic acid molecules are labeled with a detectable marker such as a radioactive or a fluorescent marker prior to hybridization. After sequence-specific hybridization of a portion of the nucleic acid molecules (target and surface-bound probe), the presence, and/or levels of a sequence of interest is measured using the radioactive or fluorescent marker.

Error in the measurement of the amounts of nucleic acid molecules having sequences of interest occurs when target-target or probe-probe intermolecular cross-hybridization affects the measurement of target-to-probe hybridization. The problem of intermolecular cross-hybridization is the undesirable binding of target-to-target or probe-to-probe nucleic acid molecules to each other, and affects all analytical methods that are based upon the specific binding of complementary nucleic acid sequences. Examples of intermolecular cross-hybridization include binding between two nucleic acid molecules with a low degree of complementary sequences and binding of a target sequence to another target sequence which already bound to a probe. Therefore, intermolecular cross-hybridization can lead to inaccurate measurements resulting from a target binding to a probe through an intermediate molecule instead of hybridizing directly to the probe.

The control of intermolecular cross-hybridization is particularly important for methods that employ massively parallel arrays of hybridization probes (DNA microarray methods). Conventional arrays depend solely upon hybridization for specificity since there is no enzyme-based proofreading of duplexes as in methods based upon Sanger dideoxy sequencing or the polymerase chain reaction, such as quantitative PCR. In addition, the large number of probes reduces the ability to verify the specificity of all probe-target interactions.

Furthermore, support-based methods for determining the presence and/or amounts of nucleic acid molecules containing nucleotide sequences of interest consume large quantities of label compared to the number of nucleic acid molecules detected. Conventional nucleic acid detection methods label all nucleic acid molecules in a sample and/or on an array, thereby also labeling nucleic acids not of interest which are discarded.

### SUMMARY

The Microarray Nuclease Protection Assay includes a method for microarray analysis of a sample for a target nucleic acid sequence including selecting a microarray comprising a set of oligonucleotide probes, contacting the set of oligonucleotide probes with a sample suspected of containing a target nucleic acid sequence under hybridization conditions to form a set of probe-target hybrids, contacting the set of probe-target hybrids with one or more single-stranded nucleases, removing the target nucleic acids from the remaining set of probe-target hybrids to expose the oligonucleotide probes remaining on the microarray, and labeling the remaining oligonucleotide probes, and analyzing the microarray for oligonucleotide probes hybridized to the target nucleic acid sequences.

Inclusion of nuclease treatment in conjunction with Microarray assay techniques increases stringency for detection of hybridized target-probe complexes by digesting unhybridized and improperly hybridized targets and/or probes. Inclusion of a target removal step simplifies and reduces reagent amounts for detection of probes which hybridized to targets. In various embodiments of the present assay, non-specific background and/or cross-hybridization is reduced, thereby improving sequence detection.

In an embodiment, the Microarray Nuclease Protection Assay includes target nucleic acids that are RNA and DNA probes. The hybridized array is digested with a single-stranded DNA 5′ exonuclease. The digestion removes any probes from the array that are not protected by strong, specific hybridization to an RNA target from the sample. The hybridized array is washed or treated to remove the 5′ exonuclease. The hybridized RNA targets are removed from the array surface (away from the remaining probes) and the remaining unhybridized probes on the array are detected.

### DETAILED DESCRIPTION

### Table of Contents

### A. Terminology

### B. Microarray Nuclease Protection Assay

### C. Nucleases for the Microarray Nuclease Protection Assay

### D. Hybridization Methods for Microarray Nuclease Protection Assay

### E. Detection Methods for Microarray Nuclease Protection Assay

### F. Arrays for Microarray Nuclease Protection Assay

### A. Terminology

Various embodiments of the present invention will be described in detail with reference to the drawings, wherein like reference numerals represent like parts throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

The term “polynucleotide” or “nucleic acid” refers to a compound or composition that is a polymeric nucleotide or nucleic acid polymer. The polynucleotide may be a natural compound or a synthetic compound. The polynucleotide can have from about 2 to 5,000,000 or more nucleotides. The larger polynucleotides are generally found in the natural state. In an isolated state the polynucleotide can have about 10 to 50,000 or more nucleotides, usually about 100 to 20,000 nucleotides. It is thus obvious that isolation of a polynucleotide from the natural state often results in fragmentation. It may be useful to fragment longer target nucleic acid sequences, particularly RNA, prior to hybridization to reduce competing intramolecular structures (e.g., cross-hybridization).

Polynucleotides include nucleic acids and fragments thereof, from any source in purified or unpurified form including deoxyribonucleic acid (DNA, dsDNA and ssDNA), ribonucleic acid (RNA), including tRNA, mRNA, rRNA, mitochondrial DNA and RNA, chloroplast DNA and RNA, DNA/RNA hybrids, or mixtures thereof, genes, chromosomes, plasmids, cosmids, the genomes of biological material such as microorganisms, e.g., bacteria, yeasts, phage, chromosomes, viruses, viroids, molds, fungi, plants, animals, humans, and the like. Polynucleotides may be purified, in mixtures with other polynucleotides, or present only as a minor fraction of a complex mixture such as a biological sample. Also included are genes, such as hemoglobin gene for sickle-cell anemia, cystic fibrosis gene, oncogenes, cDNA, and the like.

Polynucleotides can be obtained from various biological materials by procedures well known in the art. A Polynucleotide, where appropriate, may be cleaved to obtain a fragment that contains a target nucleotide sequence, for example, by shearing or by treatment with a restriction endonuclease or other site-specific chemical cleavage method.

The phrase “target nucleic acid” refers to nucleotides to be identified, detected, quantified, sequence determined or otherwise analyzed, usually existing within a portion or all of a polynucleotide. In various embodiments, the identity of the target nucleotide sequence is known to an extent sufficient to allow preparation of various sequences hybridizable with the target nucleotide sequence and of oligonucleotides, such as probes and primers, and other molecules necessary for conducting methods in accordance with the present invention, related methods and so forth.

The target nucleic acids are generally derived from a biological source, although in vitro produced or synthetic nucleic acids may also be analyzed by the inventive methods. In some embodiments, the target nucleic acids are present in a complex mixture of biological materials. The target nucleic acids may be unpurified, partially purified, or substantially purified from such complex mixtures, such as cell lysates, tissues, or blood. In some embodiments, the target nucleic acids are generated by in vitro replication and/or amplification methods such as the Polymerase Chain Reaction (PCR), asymmetric PCR, the Ligase Chain Reaction (LCR), transcriptional amplification by an RNA polymerase, and so forth from nucleic acid templates derived from biological sources.

The nucleic acids may be either single-stranded or double-stranded. A double-stranded nucleic acid may be treated to render it denatured or single stranded by treatments that are well known in the art and include, for instance, heat or alkali treatment, or enzymatic digestion of one strand. In many embodiments, double-stranded target nucleic acids are denatured prior to application and hybridization to the microarray.

The target sequence usually contains from about 10 to 5,000 or more nucleotides, preferably 50 to 1,000 nucleotides. In some embodiments, the target nucleotide sequence is a fraction of a larger molecule, while in others it may be substantially the entire molecule.

It is to be noted that the usage of the terms “probe” and “target” in the literature may vary. In the present disclosure, the term “probe” is used to refer to an immobilized or surface-bound species, and the term target is used to refer to a species in solution (the “target” of the assay). Definition of “probe” and “target” in descriptions of non-homogeneous diagnostic assays is typically consistent with the use herein. Such usage of the terms is the opposite of the usage sometimes seen in the molecular biology literature.

The term “oligonucleotide” refers to a polynucleotide, usually single stranded, either a synthetic polynucleotide or a naturally occurring polynucleotide. The length of an oligonucleotide is generally governed by the particular role thereof, such as, for example, probe, primer and the like. Various techniques can be employed for preparing an oligonucleotide. Such oligonucleotides can be obtained by biological synthesis or by chemical synthesis. For short oligonucleotides (up to about 100 nucleotides), chemical synthesis will frequently be more economical as compared to biological synthesis. In addition to economy, chemical synthesis provides a convenient way of incorporating low molecular weight compounds and/or modified bases during specific synthesis steps.

Furthermore, chemical synthesis is very flexible in the choice of length and region of the target polynucleotide binding sequence. The oligonucleotide can be synthesized by standard methods such as those used in commercial automated nucleic acid synthesizers. Chemical synthesis of DNA on a suitably modified glass or resin can result in DNA covalently attached to the surface. This may offer advantages in washing and sample handling. Methods of oligonucleotide synthesis include phosphotriester and phosphodiester methods (Narang, et al. (1979) Meth. Enzymol 68:90) and synthesis on a support (Beaucage, et al. (1981) Tetrahedron Letters 22:1859-1862) as well as phosphoramidite techniques (Caruthers, M. H., et al., “Methods in Enzymology,” Vol. 154, pp. 287-314 (1988)) and others described in “Synthesis and Applications of DNA and RNA,” S. A. Narang, editor, Academic Press, New York, 1987, and the references contained therein. The chemical synthesis via a photolithographic method of spatially addressable arrays of oligonucleotides bound to glass surfaces is described by A. C. Pease, et al., Proc. Nat. Acad. Sci. USA (1994) 91:5022-5026. Chemical synthesis of spatially addressable arrays via inkjet printing methods is described by Blanchard (Blanchard, A. P., R. J. Kaiser, et al. (1996). “High-density oligonucleotide arrays.” Biosensors & Bioelectronics 11(6/7): 687-690) and by Kronick (Kronick, M. N. (2004). “Creation of the whole human genome microarray.” Expert Rev Proteomics 1(1): 19-28).

Oligonucleotides may be employed, for example, as oligonucleotide probes or primers. The term “oligonucleotide probe” refers to an oligonucleotide employed to bind to a portion of a polynucleotide such as another oligonucleotide or a target nucleotide sequence. The design, including the length, and the preparation of the oligonucleotide probes are generally dependent upon the sequence to which they bind. Usually, the oligonucleotide probes are at least about 2 nucleotides, preferably, about 5 to about 100 nucleotides, more preferably, about 20 to about 70 nucleotides, and usually, about 30 to about 60 nucleotides, in length. The term “oligonucleotide primer(s)” refers to an oligonucleotide that is usually employed in a chain extension on a polynucleotide template such as in, for example, an amplification of a nucleic acid.

The term “nucleotide” or “nucleotide base” or “base” refers to a base-sugar-phosphate combination that is the monomeric unit of nucleic acid polymers, i.e., DNA and RNA. The term as used herein includes modified nucleotides. In general, the term refers to any compound containing a cyclic furanoside-type sugar (.beta.-D-ribose in RNA and .beta.-D-2′-deoxyribose in DNA), which is phosphorylated at the 5′ position and has either a purine or pyrimidine-type base attached at the C-1′ sugar position via a P-glycosol C1′-N linkage. The nucleotide may be natural or synthetic. The term “nucleoside” refers to a base-sugar combination or a nucleotide lacking a phosphate moiety.

The term “complementary,” “complement,” or “complementary nucleic acid sequence” refers to the nucleic acid strand that is related to the base sequence in another nucleic acid strand by the Watson-Crick base-pairing rules. In general, two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. RNA sequences can also include complementary G/U or U/G basepairs. Analogs and derivatives of the nucleic acids follow the base-pairing based upon their structural relationship.

The term “related sequences” refers to sequences having a variation in nucleotides such as in a “mutation,” for example, single nucleotide polymorphisms (SNPs). In general, the variations occur from individual to individual. The mutation may be a change in the sequence of nucleotides of normally conserved nucleic acid sequence resulting in the formation of a mutant as differentiated from the normal (unaltered) or wild-type sequence. Point mutations (i.e. mutations at a single base position) can be divided into two general classes, namely, base-pair substitutions and frameshift mutations. The latter entail the insertion or deletion of a nucleotide pair. Mutations that insert or delete multiple base pairs are also possible; these can leave the translation frame unshifted, permanently shifted, or shifted over a short stretch of sequence. A difference of a single nucleotide can be significant so to change the phenotype from normality to abnormality as in the case of, for example, sickle cell anemia.

The term “substrate” as used herein refers to a surface upon which marker molecules or probes, e.g., a microarray, may be adhered. Glass slides are the most common substrate for biochips, although fused silica, silicon, plastic, flexible web and other materials are also suitable.

The term “sensitivity” refers to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of: the nature of the surface immobilized nucleic acids, the nature of the hybridization and wash conditions, the nature of the labeling system, the nature of the detection system, etc.

The term “specificity” refers to the ability of one member of a pair of binding molecules to differentiate between similar molecules and associate only with its given partner

### Additional definitions are provided within the context of the disclosure provided below.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

### B. Microarray Nuclease Protection Assay

The Microarray Nuclease Protection Assay includes hybridizing target nucleic acids to nucleic acid probes on an array, a single-stranded nuclease treatment step to digest unhybridized and improperly hybridized probes and/or targets, followed by target nucleic acid removal from any remaining hybridized probes and/or targets, and detection of the remaining probes on the array.

In further embodiments, the Microarray Nuclease Protection Assay includes a method for microarray analysis of a sample for a target nucleic acid sequence including selecting a microarray comprising a set of oligonucleotide probes, contacting the set of oligonucleotide probes with a sample suspected of containing a target nucleic acid sequence under hybridization conditions to form a set of probe-target hybrids, contacting the set of probe-target hybrids with one or more single-stranded nucleases, removing the target nucleic acids from the remaining set of probe-target hybrids to expose the oligonucleotide probes remaining on the microarray, and labeling the remaining oligonucleotide probes. The sub-set of the original nucleic acid probes on the microarray surface are analyzed to reveal information about the target nucleic acid sequences previously hybridized to them.

Inclusion of nuclease treatment in conjunction with Microarray assay techniques increases stringency for detection of hybridized target-probe complexes by digesting unhybridized and improperly hybridized targets and/or probes. Inclusion of a target removal step simplifies and reduces reagent amounts for detection of probes which hybridized to targets. In various embodiments of the present assay, non-specific background and/or cross-hybridization is reduced, thereby improving sequence detection.

Microarray nuclease protection assay find use in a variety of different applications. For example, hybridization assays or nucleic acid detection applications in which the presence of a particular target nucleic acid sequence in a given sample is detected. In various embodiments, the target nucleic acid is at least qualitative, and in further embodiment quantitative. The Microarray Nuclease Protection Assay is suitable for use with various conventional hybridization assays of interest including: gene discovery assays, differential gene expression analysis assays; nucleic acid sequencing assays, and the like. Patents and patent applications describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference.

The Nuclease Protection assay includes a single-stranded nuclease treatment and target removal step. The hybridized array is first contacted with a single-stranded nuclease treatment as described above to digest unduplexed probes from the array surface. After digestion, only those oligonucleotide probes protected by hybridization with a target nucleotide remain attached to the array surface. Typically, the array is washed to remove the single-stranded nuclease and any digestion products from the array.

Subsequently, a target removal step is performed. In the target removal step, the targets of the target:probe hybrids on the array are removed from the array surface. In one embodiment, a target-selective nuclease treatment is used to digest the targets away from the remaining duplexed probe/targets on the array. In another embodiment, the targets are denatured away from the probes. In further embodiments, denaturation of targets from the target:probe hybrids is by either or both heat or chemical means, for example, but not limited to high concentrations or chaotropic salts or solvents. In various embodiments, target removal includes washing the array. After target removal, a sub-set of the original oligonucleotide probes complementary to the targets remains attached to the array. The array is washed to remove the RNAse and prepare for detection by any of a variety of methods for detection of the remaining oligonucleotides probes.

In a further embodiment, wherein the target nucleic acid sequences contained in the sample are RNA, the oligonucleotides probes are DNA or analogs thereof. In a still further embodiment, the single-stranded nuclease treatment contacts the hybridized array with one or more nucleases for digestion of single-stranded DNA.

In an embodiment of the Microarray Nuclease Protection Assay, the target nucleic acid sequences contained in the sample are RNA. In these embodiments, the RNA containing sample is hybridized with the surface of the array including the oligonucleotides probes. Following optional wash steps, the array is contacted with a single-stranded nuclease as described above. Following optional washing, the hybridized array is treated with RNAse or a chemical RNA hydrolysis catalyst (e.g. Zn⁺² ion) to digest the RNA targets on the array, (e.g., away from the duplexes). The array is washed to remove the RNAse or chemical catalyst. The array is incubated with a mixture of end-labeled oligonucleotides that are complementary to the array-bound probes. The end-labeled oligonucleotides may be either direct (e.g., Cy3) or indirect (e.g., biotin). After hybridization, the arrays are washed (i.e. to remove unbound labeled targets). The array is further developed if required by the labeling scheme (e.g., indirect labeling methods). The array is scanned and interpreted according to known methods for conventional single-color microarrays.

In an alternative embodiment, the set of oligonucleotide probes are DNA attached to the microarray at 5′ end and the target nucleic acid sequence of the sample are also DNA. Single-stranded DNAses are used to contact the probe:target hybrids to digest unduplexed DNA oligonucleotide probes. In embodiments, where both target and probe are similar nucleic acids, for example both DNA, then the target removal step is performed by denaturation of DNA target nucleic acid away from the remaining DNA oligonucleotide probes. In further embodiments, denaturation of nucleic acids is performed by either heat or chemical means or both, as is commonly practiced in the art. Preferably, such denaturation steps are performed such that the probes bound to the array are not damaged or removed in a manner to hinder later detection.

Additional aspects of the Microarray Nuclease Protection Assay are described in further detail below.

### C. Nucleases

One or more nuclease treatments are included in the Microarray Nuclease Protection Assay. At least one nuclease treatment of the array, which may also be referred to as a “single-stranded nuclease treatment,” occurs after hybridization of sample believed to contain target nucleotide sequence to the oligonucleotide probe(s). “Hybridized array” refers to the array after contact of a sample believed to contain target nucleotide sequence to the oligonucleotide probe(s) under conditions suitable for hybridization of complementary target and probe sequences.

Nucleases suitable for use in the single-stranded nuclease treatment are nucleases that preferentially digest single-stranded nucleic acids, referred to herein as “single-stranded nucleases.” The single-stranded nucleases can be non-specific, digesting both RNA and DNA, and/or variants thereof. In other embodiments, the single-stranded nucleases preferentially digest single-stranded RNA, i.e. “single-stranded RNases,” or preferentially digest single-stranded DNA, i.e. “single-stranded DNases.” Finally, nucleases may digest only from the 5′-end of a polynucleotide, i.e. 5′ single-stranded nucleases, the 3′-end of a polynucleotide, i.e. 3′ single-stranded nucleases, or both ends of a single-stranded polynucleotide.

In an embodiment, the nuclease digests single-stranded (i.e. unduplexed) nucleic acids of the oligonucleotide probes and target nucleotides. In an alternative embodiment, the nuclease selectively digests the single-stranded nucleic acids of the target nucleotides. In yet another embodiment, the nuclease selectively digests the single-stranded nucleic acids of the oligonucleotide probes. The specific nuclease selected for nuclease treatment depends on the desired selectivity (e.g., probe and/or target digestion) and nature of the nucleic acids making up the probe and target.

The single-stranded nuclease treatment is used to improve the quality of hybridization on the microarrays. More particularly, after the array of oligonucleotides has been combined with a labeled target nucleic acid to form target-oligonucleotide hybrid complexes, the target-oligonucleotide hybrid complexes are treated with a nuclease, and in most embodiments, subsequently washed to remove the nuclease and digested fragments of probe from non-perfectly complementary target-oligonucleotide hybrid complexes. Following nuclease treatment, the target:oligonucleotide hybrid complexes which are perfectly complementary remain on the array surface and are more readily identified. From the location of the labeled targets, the oligonucleotide probes which hybridized with the targets can be identified and, in turn, the sequence and/or quantity or other information about the target nucleic acid can more readily be determined or verified.

In an embodiment, the target is RNA, and the probe is DNA, a single-stranded DNA nuclease is used for the single-stranded nuclease. In an alternative embodiment, if the target is DNA and the probe is RNA, a RNA nuclease is used. In yet another embodiment, the target is DNA and the probe is DNA or synthetic derivatives thereof, and a DNA nuclease is used for the single-stranded nuclease digestion. RNase A is an example of an RNA nuclease that can be used to remove single-stranded RNA from a microarray surface. RNase A effectively recognizes and cuts single-stranded RNA, including RNA in RNA:DNA hybrids that is not in a perfect double-stranded structure. Moreover, RNA bulges, loops, and even single base mismatches can be recognized and cleaved by RNase A. In addition, RNase A recognizes and cleaves target RNA which binds to multiple oligonucleotide probes present on the substrate if there are intervening single-stranded regions. S1 nuclease and Mung Bean nuclease are examples of DNA nucleases with similar properties for single-stranded DNA. Single-stranded nucleases for use in the Microarray Nuclease Protection Assay include, but are not limited to, those listed in Table 1.

TABLE 1
Examples of single-stranded nucleases

| | Nuclease preferentially | |
|---|---|---|
| Single-stranded nucleases | digests: | Supplier |
| S1 nuclease | single-stranded DNA | multiple sources¹ |
| | single-stranded RNA | |
| Mung-bean nuclease | single-stranded DNA | multiple sources¹ |
| Ribonuclease A | single-stranded RNA | multiple sources¹ |
| RNAse T1 | single-stranded RNA | multiple sources¹ |
| Exonuclease I | Single-stranded | multiple sources¹ |
| | DNA 3′→5′ | |
| RNase ONE ™ | single-stranded RNA | Promega Corporation |

¹Example suppliers: New England Biolabs, Ipswich, MA; Promega Corporation, Madison, WI; and Applied Biosystems, Foster City, CA.

A number of nucleases are commercially available for use with the Microarray Nuclease Protection Assay and may be used either alone or in combination. For example, S1 nuclease degrades single stranded DNA or RNA from the 5′-end. Duplexed DNA, duplexed RNA, and DNA:RNA hybrids are relatively resistant to the enzyme. The enzyme is also known to be more active on DNA than RNA. Mung-bean nuclease degrades single-stranded DNA from both ends. Duplexed DNA, duplexed RNA, and DNA:RNA hybrids are relatively resistant to the enzyme. Ribonuclease A is an endoribonuclease that specifically digests single-stranded RNA 3′ to pyrimidine residues. RNase T1 is an endoribonuclease that specifically attacks the 3′ phosphate groups of guanine nucleotides and cleaves the 5′-phosphate linkage to the adjacent nucleotide. Divalent zinc cation (Zn⁺²) is a chemical catalyst that hydrolyzes the phosphosiester bonds of RNA, but not those of DNA. General guidance regarding use of each nuclease, such as amount of enzyme and buffer requirements, to achieve digestion of a given amount single-stranded nucleotides is generally provided by the supplier.

In embodiments of the Microarray Nuclease Protection Assay where target removal includes target-selective nuclease treatment, the particular nuclease used for the target-selective nuclease treatment depends on the target nucleic acid being removed from the array surface and the probe to remain on the surface of the microarray. In these embodiments, the target nucleic acids and the probe nucleic acids are have sufficient structural differences to allow for selective digestion of the targets. The probes are not digested, and are preferably not damaged in the target removal step.

In one example, the target is RNA, and the remaining probes are DNA, a RNA nuclease is used for target removal. Similarly, if the target is DNA and the probes are RNA or modified DNA that is nuclease resistant, a DNA nuclease is used. In further embodiments, denaturation of the target nucleic acids from the target:probe hybrids is used in combination with nuclease treatment for target removal. The target removal steps may also include one or more washing steps.

### D. Hybridization Methods

The Microarray Nuclease Protection Assay utilizes hybridization of a target nucleic acid sequence to oligonucleotide probes in order to select duplexed probes for later detection. In some embodiments, the hybridization of target to probe protects the duplex from nuclease degradation. In some embodiments, a set of oligonucleotide probes on the microarray are target-specific, meaning that the probes were selected for their sequence relationship to the target nucleic acid or acids of interest. In other embodiments, the oligonucleotide probes are part of a tiling array which represents a range of nucleic acid sequences. For example, a genome or portion thereof. The terms “hybridization (hybridize)” and “binding” are used interchangeably and refer to the process by which single strands of nucleic acid sequences form double-helical segments through hydrogen bonding between complementary nucleotides. “Hybrid” or “duplex,” also used interchangeably herein, refer to a double-stranded nucleic acid molecule formed by hybridization.

The ability of two nucleotide sequences to hybridize with each other is based on the degree of complementarity of the two nucleotide sequences, which in turn is based on the fraction of matched complementary nucleotide pairs. The more nucleotides in a given sequence that are complementary to another sequence, the more stringent the conditions can be for hybridization and the more specific will be the binding of the two sequences. Increased stringency of binding is achieved by elevating the temperature, increasing the ratio of co-solvents, lowering the salt concentration, and the like. The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions.

In the Microarray Nuclease Protection Assay, the degree of stringency of hybridization of the duplex of target to probe is also influenced by the nuclease. Improperly hybridized nucleic acids, for example with low degree of complementarity, looped out regions, or cross-hybridization are likely to be digested by the single-stranded nucleases. In various embodiments, the nuclease acts as an enzyme-based proofreader of the duplexed target to probe. Poorly hybridized pairs and cross-hybridized nucleotides are digested by the nuclease. The result is greater stringency of hybridization over non-nuclease challenged hybridizations. The use of nuclease provides an additional means for checking or providing stringent and/or specific hybridization.

In various embodiments, the array hybridization conditions are controlled for specific or selective hybridization of target nucleic acids, including related sequences to probes on the array. Specific or selective hybridization refers to the binding, duplexing, or hybridizing of one or more target nucleic acid molecules preferentially to particular complementary nucleotide sequence(s) on the array under stringent conditions. In the Microarray Nuclease Hybridization Assay, a combination of one or more nuclease treatments and optionally other stringent hybridization and/or wash conditions are applied to control the stringency of hybridization of target to probe.

Stringent hybridization conditions as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent hybridization conditions are the summation or combination (totality) of both hybridization solvation conditions, nuclease treatment, and wash conditions.

A stringent hybridization and stringent hybridization wash conditions in the context of nucleic acid hybridization (e.g., as in array, Southern or Northern hybridizations) are sequence dependent, and are different under different experimental parameters. Stringent hybridization conditions that can be used to identify nucleic acids within the scope of the invention can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1 M NaCl, 0.5% sodium sarcosine, 50 mM MES, pH 6.5. Those of ordinary skill will readily recognize that alternative but comparable hybridization and wash conditions can be utilized to provide conditions of similar stringency.

In certain embodiments, the stringency of the wash conditions that set forth the conditions which determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

A specific example of stringent assay conditions is rotating hybridization at 65° C. in a salt based hybridization buffer with a total monovalent cation concentration of 1.5 M (e.g., as described in U.S. patent application Ser. No. 09/655,482 filed on Sep. 5, 2000, the disclosure of which is herein incorporated by reference) followed by washes of 0.5×SSC and 0.1×SSC at room temperature.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no more” is meant less than about 5-fold more, typically less than about 3-fold more. Other stringent hybridization conditions are known in the art and may also be employed, as appropriate.

### E. Detection Methods

The Microarray Nuclease Protection Assay utilizes hybridization of a target nucleic acid sequence to a oligonucleotide probe in combination with nuclease treatment and removal of the target oligonucleotide from the duplexed probe to select probes on an array for detection, thereby eliciting information about the target nucleic acid sequence. In one embodiment, detecting the remaining oligonucleotide probes is performed by hybridizing oligonucleotide primers complementary to the probes near the distal end (i.e. the end distal to the array surface, commonly the 5′ end, but may be the 3′), to leave a single-stranded overhang. The hybridized oligos are extended at least one base using standard dideoxy-dNTP end labeling techniques. The array is washed to remove excess dNTP reagents and polymerase. The array is read and interpreted according to conventional methods.

In another embodiment, the method of detection of probes remaining on the array is contacting the microarray surface with a mixture of previously end-labeled oligonucleotides that are complementary to the remaining oligonucleotide probes. The end-labeled oligonucleotides may be suitable for either direct (e.g., Cy3) or indirect (e.g., biotin) detection. After hybridization, the arrays are washed (i.e. to remove unbound labeled oligonucleotides). The array is further developed if required by the labeling scheme (e.g., indirect labeling methods). The array is scanned and interpreted according to known methods for conventional single-color microarrays.

In yet another embodiment, the original array-bound probe oligonucleotide sits atop an oligonucleotide stilt containing an easily labeled nucleotide analog, such as aminoallyl dU. In this case, fluorophore labeling is performed using an amino-reactive fluorophore (e.g. “active ester”) well known to the art. In a variant of this embodiment, the modified nucleotide exposes a ligand for non-covalent labeling (e.g. biotin); a fluorophore is then added via binding of a labeled macromolecule (e.g. strepavidin) that specifically recognizes the ligand.

In further embodiments of any of the previous embodiments, the remaining DNA probes for detection are associated (e.g., via extension or hybridization) with a fluorescent label to the probe and where analyzing the DNA probes comprises obtaining a quantitative fluorescence image of said DNA probes remaining on the microarray.

### Labels

The remaining probes on the array surface can be labeled with any of a number of convenient detectable markers through various direct or indirect methods. Detectable markers include chromogens, radioisotopes, chemiluminescent compounds, labeled binding proteins, heavy metal atoms, spectroscopic markers, magnetic labels, and linked enzymes. Suitable chromogens which can be employed include those molecules and compounds which adsorb light in a distinctive range of wavelengths so that a color can be observed or, alternatively, which emit light when irradiated with radiation of a particular wave length or wave length range, e.g., fluorescers.

A wide variety of suitable dyes are available, being primary chosen to provide an intense color with minimal absorption by their surroundings. Illustrative dye types include quinoline dyes, triarylmethane dyes, acridine dyes, alizarine dyes, phthaleins, insect dyes, azo dyes, anthraquinoid dyes, cyanine dyes, phenazathionium dyes, and phenazoxonium dyes.

A wide variety of fluorescers can be employed either by alone or, alternatively, in conjunction with quencher molecules. Fluorescers of interest fall into a variety of categories having certain primary functionalities. These primary functionalities include 1- and 2-aminonaphthalene, p,p′-diaminostilbenes, pyrenes, quaternary phenanthridine salts, 9-aminoacridines, p,p′-diaminobenzophenone imines, anthracenes, oxacarbocyanine, merocyanine, 3-aminoequilenin, perylene, bisbenzoxazole, bis-p-oxazolyl benzene, 1,2-benzophenazin, retinol, bis-3-aminopyridinium salts, hellebrigenin, tetracycline, sterophenol, benzimidzaolylphenylamine, 2-oxo-3-chromen, indole, xanthen, 7-hydroxycoumarin, phenoxazine, salicylate, strophanthidin, porphyrins, triarylmethanes and flavin. Individual fluorescent compounds which have functionalities for linking or which can be modified to incorporate such functionalities include, e.g., dansyl chloride; fluoresceins such as 3,6-dihydroxy-9-phenylxanthhydrol; rhodamineisothiocyanate; N-phenyl 1-amino-8-sulfonatonaphthalene; N-phenyl 2-amino-6-sulfonatonaphthalene: 4-acetamido-4 isothiocyanato-stilbene-2,2′-disulfonic acid; pyrene-3-sulfonic acid; 2-toluidinonaphthalene-6-sulfonate; N-phenyl, N-methyl 2-aminoaphthalene-6-sulfonate; ethidium bromide; stebrine; auromine-0,2-(9′-anthroyl)palmitate; dansyl phosphatidylethanolamine; N,N′-dioctadecyl oxacarbocyanine; N,N′-dihexyl oxacarbocyanine; merocyanine, 4(3′pyrenyl)butyrate; d-3-aminodesoxy-equilenin; 12-(9′anthroyl)stearate; 2-methylanthracene; 9-vinylanthracene; 2,2′(vinylene-p-phenylene)bisbenzoxazole; p-bis[2-(4-methyl-5-phenyl-oxazolyl)]benzene; 6-dimethylamino-1,2-benzophenazin; retinol; bis(3′-aminopyridinium) 1,10-decandiyl diiodide; sulfonaphthylhydrazone of hellibrienin; chlorotetracycline; N(7-dimethylamino-4-methyl-2-oxo-3-chromenyl)maleimide; N-[p-(2-benzimidazolyl)-phenyl]maleimide; N-(4-fluoranthyl)maleimide; bis(homovanillic acid); resazarin; 4-chloro-7-nitro-2,1,3benzooxadiazole; merocyanine 540; resorufin; rose bengal; and 2,4-diphenyl-3(2H)-furanone.

Desirably, fluorescers should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Fluorescers are generally preferred because by irradiating a fluorescer with light, one can obtain a plurality of emissions. Thus, a single label can provide for a plurality of measurable events.

Detectable signal can also be provided by chemiluminescent and bioluminescent sources. Chemiluminescent sources include a compound which becomes electronically excited by a chemical reaction and can then emit light which serves as the detectible signal or donates energy to a fluorescent acceptor. A diverse number of families of compounds have been found to provide chemiluminescence under a variety or conditions. One family of compounds is 2,3-dihydro-1,4-phthalazinedione. The must popular compound is luminol, which is the 5-amino compound. Other members of the family include the 5-amino-6,7,8-trimethoxy- and the dimethylamino[ca]benz analog. These compounds can be made to luminesce with alkaline hydrogen peroxide or calcium hypochlorite and base. Another family of compounds is the 2,4,5-triphenylimidazoles, with lophine as the common name for the parent product. Chemiluminescent analogs include para-dimethylamino and -methoxy substituents. Chemiluminescence can also be obtained with oxalates, usually oxalyl active esters, e.g., p-nitrophenyl and a peroxide, e.g., hydrogen peroxide, under basic conditions. Alternatively, luciferins can be used in conjunction with luciferase or lucigenins to provide bioluminescence.

### Reading the Array

In various embodiments, sequence detection of the array includes: labeling the remaining oligonucleotide probes and reading the array. Reading of the array may be accomplished, for example, by illuminating the array and reading the location and intensity of resulting fluorescence at each feature of the array to detect any binding complexes on the surface of the array. For example, a scanner may be used for this purpose which is similar to the AGILENT MICROARRAY SCANNER available from Agilent Technologies, Palo Alto, Calif. Other suitable apparatus and methods are described in U.S. patent applications: Ser. No. 09/846,125 “Reading Multi-Featured Arrays” by Dorsel et al.; and Ser. No. 09/430,214 “Interrogating Multi-Featured Arrays” by Dorsel et al. As previously mentioned, these references are incorporated herein by reference. However, arrays may be read by any other method or apparatus than the foregoing, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,221,583 and elsewhere). Results from the reading may be raw results (such as fluorescence intensity readings for each feature in one or more color channels) or may be processed results such as obtained by rejecting a reading for a feature which is below a predetermined threshold and/or forming conclusions based on the pattern read from the array (such as whether or not a particular target sequence may have been present in the sample or an organism from which a sample was obtained exhibits a particular condition). The results of the reading (processed or not) may be forwarded (such as by communication) to a remote location if desired, and received there for further use (such as further processing).

In certain embodiments, the subject methods include a step of transmitting data from at least one of the detecting and deriving steps, as described above, to a remote location. By “remote location” is meant a location other than the location at which the array is present and hybridization occur. For example, a remote location could be another location (e.g. office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### F. Arrays for Microarray Nuclease Protection Assay

In the Microarray Nuclease Protection Assay of the present invention, an array of diverse oligonucleotides at known locations on a single substrate surface is employed.

The term “array” encompasses the term “microarray” and refers to an ordered array presented for binding to nucleic acids and the like. Arrays are generally made up of a plurality of distinct or different features. The term “feature” is used interchangeably herein with the terms: “features,” “feature elements,” “spots,” “addressable regions,” “regions of different moieties,” “surface or substrate immobilized elements” and “array elements,” where each feature is made up of oligonucleotides bound to a surface of a solid support, also referred to as substrate immobilized nucleic acids.

An “array,” includes any one-dimensional, two-dimensional or substantially two-dimensional (as well as a three-dimensional) arrangement of addressable regions bearing a particular chemical moiety or moieties. In the present disclosure, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus).

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one which is to be evaluated by the other (thus, either one could be an unknown mixture of analytes, e.g., polynucleotides, to be evaluated by binding with the other).

As indicated above, the arrays are arrays of nucleic acids, including oligonucleotides, polynucleotides, DNAs, RNAs, synthetic mimetics thereof, and the like. The subject arrays include at least two distinct nucleic acids that differ by monomeric sequence immobilized on, e.g., covalently to, different and known locations on the substrate surface. In certain embodiments, each distinct nucleic acid sequence of the array is typically present as a composition of multiple copies of the polymer on the substrate surface, e.g., as a spot on the surface of the substrate. The number of distinct nucleic acid sequences, and hence spots or similar structures, present on the array may vary, but is generally at least 2, usually at least 5 and more usually at least 10, where the number of different spots on the array may be as a high as 50, 100, 500, 1000, 10,000 or higher, depending on the intended use of the array. The spots of distinct polymers present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the polymeric sequences are not arranged in the form of distinct spots, but may be positioned on the surface such that there is substantially no space separating one polymer sequence/feature from another.

Arrays can be fabricated using drop deposition from pulsejets of either polynucleotide precursor units (such as monomers) in the case of in situ fabrication, or the previously obtained polynucleotide. Such methods are described in detail in, for example, the previously cited references including U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication, as previously described herein.

An “array layout” refers to one or more characteristics of the features, such as feature positioning on the substrate, one or more feature dimensions, and an indication of a moiety at a given location. A “scan region” refers to a contiguous (preferably, rectangular) area in which the array spots or features of interest, as defined above, are found. The scan region is that portion of the total area illuminated from which the resulting fluorescence is detected and recorded. For the purposes of this invention, the scan region includes the entire area of the slide scanned in each pass of the lens, between the first feature of interest, and the last feature of interest, even if there are intervening areas which lack features of interest.

### G. Kits for performing Microarray Nuclease Protection Assay

A kit containing materials for performing the Microarray Nuclease Protection Assay of the present invention is also envisioned. In one embodiment, a kit includes one or more single-stranded nucleases, a means for removal of RNA from the probe:target hybrids; and one or more labeling moieties for labeling DNA oligonucleotide probes. In a further embodiment, the kit also includes instructions for use of the nucleases, RNases and labeling moieties on a microarray for performing the Microarray Nuclease Protection Assay of the present invention of a sample for a target nucleic acid sequence.

In a further embodiment, a kit contains materials specific for single-stranded nucleases digestion of DNA. In another embodiment, a kit contains means for removal of RNA by RNase digestion, for example one or more RNases. In another embodiment, a kit contains means for removal of RNA by catalytic hydrolysis, for example Zn2+ ions. In a still further embodiment of any of the above kits includes an array of diverse oligonucleotides at known locations on a single substrate surface, for example, a microarray. In additional further embodiments, any of the above kits also include one or more sample buffers, hybridization buffers, and wash buffers.

All publications and patent applications in this specification are indicative of the level of ordinary skill in the art to which this invention pertains and are incorporated herein by reference in their entireties.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

The claimed invention is:

1. A method for microarray analysis of a sample for a target nucleic acid sequence, the method comprising:
selecting a microarray comprising a set of oligonucleotide probes;
contacting the set of oligonucleotide probes with a sample suspected of containing a target nucleic acid sequence under hybridization conditions to form a set of probe-target hybrids;
contacting the set of probe-target hybrids with one or more single-stranded nucleases;
removing the target nucleic acids from the remaining set of probe-target hybrids to expose the oligonucleotide probes remaining on the microarray; and
labeling the remaining oligonucleotide probes; and
analyzing the microarray for oligonucleotide probes hybridized to the target nucleic acid sequences.

2. The method of claim 1, wherein the oligonucleotide probes comprise between 30 and 100 nucleotides.

3. The method of claim 1, wherein the set of oligonucleotide probes are DNA.

4. The method of claim 3, wherein the nuclease is a DNA single-stranded nuclease and the contacting the set of oligonucleotide probes hybridized with target nucleic acid sequences with one or more single-stranded nucleases further comprises digesting unduplexed DNA oligonucleotide probes without digesting duplexed DNA oligonucleotide probes.

5. The method of claim 4, wherein the target nucleic acid sequence of the sample is RNA.

6. The method of claim 5, wherein removing the target nucleic acid sequence hybridized to the DNA target specific oligonucleotide probes is selected from heat denaturation, or chemical denaturation.

7. The method of claim 5, wherein the step of contacting the probe:target hybrids with one or more single-stranded nucleases additionally comprises digesting unduplexed DNA oligonucleotide probes; and wherein the step of removing the target nucleic acids comprises contacting the set of oligonucleotide probes hybridized with target nucleic acid sequences with one or more RNases, and digesting RNA target nucleic acid sequences away from the remaining DNA oligonucleotide probes.

8. The method of claim 5, wherein the step of contacting the probe:target hybrids with one or more single-stranded nucleases additionally comprises digesting unduplexed DNA oligonucleotide probes; and wherein the step of removing the target nucleic acids comprises contacting the set of oligonucleotide probes hybridized with target nucleic acid sequences with one or more agents for chemical catalyzed hydrolysis RNA target nucleic acid sequences away from the remaining DNA oligonucleotide probes.

9. The method of claim 5, wherein labeling comprises hybridizing a primer adjacent to distal end of DNA oligonucleotide probes; and
incorporating one or more labels by polymerase activity.

10. The method of claim 1, wherein the set of oligonucleotide probes are DNA attached to the microarray at 5′ end and the target nucleic acid sequence of the sample is DNA.

11. The method of claim 10, wherein the step of contacting the probe:target hybrids with one or more single-stranded nucleases additionally comprises digesting unduplexed DNA oligonucleotide probes; and wherein the step of removing the target nucleic acids comprises denaturation of DNA target nucleic acid away from the remaining DNA oligonucleotide probes.

12. A method for microarray analysis of one or more target RNA from a sample, the method comprising
hybridizing a sample containing one or more target RNAs to a microarray of DNA probes to form a set of target-probe hybrids;
digesting any unbound and poorly hybridized DNA probes with a single-stranded DNA nuclease;
removing target RNAs from target-probe hybrids to expose a set of DNA probes on the microarray;
labeling the DNA probes; and
analyzing the DNA probes remaining on the microarray to gain information about one or more of target RNAs.

13. The method of claim 12, wherein the oligonucleotide probes comprise between 30 and 100 nucleotides.

14. The method of claim 12, wherein the target RNAs and DNA probes are hybridized under stringent conditions.

15. The method of claim 12, wherein the target RNAs are removed from target-probe hybrids by treating with an RNA nuclease.

16. The method of claim 12, wherein labeling the DNA probes comprises associating a fluorescent label to the probe and where analyzing the DNA probes comprises obtaining a quantitative fluorescence image of said DNA probes remaining on the microarray.

17. The method of claim 12, wherein labeling the DNA probes comprises hybridizing a nucleic acid primer to the DNA probe with an overhanging 3′ end; and end-labeling said end.

18. The method of claim 12, wherein the information gained about one or more of target RNAs is quantification of the RNA in the sample.

19. The method of claim 12, wherein the information gained about one or more of target RNAs is determination of sequence information for one or more RNAs in the sample.

20. A kit for microarray analysis of a sample for a target RNA sequence on a microarray comprising a set of DNA oligonucleotide probes, the kit comprising:
one or more single-stranded DNA nucleases;
one or more RNases; and
one or more labeling moieties for labeling DNA oligonucleotide probes; and
instructions for use of the nucleases, RNases and labeling moieties on a microarray for analysis of a sample for a target RNA sequence.

21. The kit of claim 20 additionally comprising a microarray comprising a set of oligonucleotide probes.
